(19) United States
(12) Patent Application Publication
(10) Pub. No.: US 2006/0199805 A1
(43) Pub. Date: Sep. 7, 2006

(54) Pharmaceutical compositions for the treatment and/or prevention of anxiety disorders

(75) Inventors: Robert Pyke, New Fairfield, CT (US); Angelo Ceci, Mittelbiberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/364,785

(22) Filed: Feb. 28, 2006

Related U.S. Application Data
(60) Provisional application No. 60/658,611, filed on Mar. 4, 2005.

(51) Int. Cl. A61K 31/5513; A61K 31/551; A61K 31/405; A61K 31/277; A61K 31/445; A61K 31/381; A61K 31/41

(52) U.S. Cl. 514/220; 514/221; 514/419; 514/649; 514/381; 514/521

(57) ABSTRACT

The invention relates to new pharmaceutical compositions for the treatment and/or prevention of anxiety disorders and methods for the preparation thereof. In a preferred embodiment, the instant invention is directed to pharmaceutical combinations comprising flibanserin as one active ingredient in combination with at least one additional active ingredient for the treatment and/or prevention of anxiety disorders and methods for the preparation thereof.

The invention relates to new pharmaceutical compositions for the treatment and/or prevention of anxiety disorders and methods for the preparation thereof. In a preferred embodiment, the instant invention is directed to pharmaceutical combinations comprising flibanserin as one active ingredient in combination with at least one additional active ingredient for the treatment and/or prevention of anxiety disorders and methods for the preparation thereof.

### BACKGROUND OF THE INVENTION

The invention relates to new pharmaceutical compositions for the treatment and/or prevention of anxiety disorders and methods for the preparation thereof. In one embodiment, the instant invention is directed to pharmaceutical combinations comprising a therapeutically effective amount of flibanserin 1 as one active ingredient in combination with a therapeutically effective amount of one or more, preferably one additional anxiolytic 2 for the treatment and/or prevention of anxiety disorders and methods for the preparation thereof.

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure: 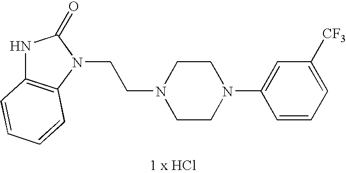
Flibanserin shows affinity for the 5-HT_{1A}_{⁻}, 5-HT₂_{⁻} and D₄-receptor. It is therefore a promising therapeutic agent for the treatment of a variety of diseases, for instance depression, schizophrenia, Parkinson, anxiety, sleep disturbances, sexual and mental disorders and age associated memory impairment.

One embodiment of the invention is directed to pharmaceutical compositions comprising a therapeutically effective amount of flibanserin 1 in combination with a therapeutically effective amount of one or more additional anxiolytics 2.

Another embodiment of the invention is directed to pharmaceutical compositions comprising a therapeutically effective amount of flibanserin 1 in combination with a therapeutically effective amount of one or more, preferably one anxiolytic 2 selected from the group consisting of benzodiazepine agonists, 5-HT_{1A} agonists, 5-HT reuptake inhibitors, chloride channel modulators, MAO-A inhibitors, GABA antagonists, 5-HT2 antagonists, monoamine reuptake inhibitors, GABA agonists, 5-HT2C antagonists, GABA-A modulators, NK1 antagonists, 5-HT1B antgonists, alpha 2 adrenoreceptor agonists, glutamate agonists, melatonin agonists, mGluR3 agonists, mGluR2 agonists, CCK2 antagonists, NK 3 antagonists and CGRP antagonists.

The compositions according to the invention may contain flibanserin 1 and the one or more additional anxiolytics 2 in a single formulation or in separate formulations. If flibanserin and the one or more additional anxiolytics are present in separate formulations these separate formulations may be administered simultaneously or sequentially.

A preferred embodiment according to the invention is directed to pharmaceutical compositions comprising a therapeutically effective amount of flibanserin 1 and a therapeutically effective amount of one or more, preferably one additional anxiolytic 2, optionally in combination with a pharmaceutically acceptable excipient.

Examples of suitable additional anxiolytics of the above mentioned compound classes include bupropion, clonazepam, alprazolam, lorazepam, clorazepate, oxazepam, flurazepam, diazepam, halazepam, prazepam, chlordiazepoxide, buspirone, gepirone, tandospirone, ipsapirone, bentazepam, citalopram, clobazam, clotiazepam, etifoxine, etizolam, delorazepam, ethyl loflazepate, flutazolam, fluoxetine, flutoprazepam, ketazolam, metaclazepam, mexazolam, moclobemide, oxazolam, tofisopam, pinazepam, paroxetine, pivagabine, rilmazafone, sertraline, tianeptine, venlafaxine, zotepine, escitalopram, fluvoxamine, pregabalin (PD-144723), agomelatine, duloxetine, LY-544344 (Elly Lilly), ocinaplon, pagoclone, aprepitant, dexmedetomidine, eglumegad, POL-240 (1H-indole-3-pyruvic acid), eplivanserin, vestipitant, levetiracetam, MKC-242 (Medici Nova), olanzapine, R-673 (Roche), SL-651498 (Sanofi-Aventis), SLV-308 (Solvay), tiagabine, ABT-089 (Abbott), emapunil, dextofisopam, itriglumide, S-desmethylzopiclone, SSR-146977 (Sanofi-Aventis), SSR-149415 (Sanofi-Aventis), gabapentin, opipramol, sumatriptan, TPA-023 (L 838417, Merck & Co.) and nefazodone, optionally in form of the pharmaceutically acceptable acid addition salts, in form of the hydrates and/or solvates and optionally in the form of the individual optical isomers, mixtures of the individual enantiomers or racemates thereof.

Preferred additional anxiolytics 2 include fluoxetine and buspirone, optionally in form of the pharmaceutically acceptable salts, in form of the hydrates and/or solvates and optionally in the form of the individual optical isomers, mixtures of the individual enantiomers or racemates thereof.

Flibanserin 1 may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin 1 is used in form of the free base, it is preferably used in form of flibanserin polymorph A as disclosed in WO 03/014079.

The anxiolytics 2 which are suitable to be combined with flibanserin within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

As used herein, the term “anxiolytic” refers to a drug suitable to relieve anxiety and reduce tension, irritability, distress, restlessness, fears, phobias, avoidance behaviors, worry, compulsions, obsessions, flashbacks, increased arousal, difficulty concentrating, hypervigilance, startle response, and somatic symptoms of anxiety including palpitations, sweating, trembling, shortness of breath, choking feelings, chest pain, nausea, dizziness, lightheadedness, faintness, derealization, depersonalization, paresthesias, chills or hot flushes, being easily fatigued, muscle tension and insomnia.

In the present invention the term “modulator” means compounds that produce tissue specific effects that can be agonistic or antagonistic.

As used herein, the term “anxiety disorder” refers to an emotional and/or behavioural disturbance characterized by persistent and pervasive worry or restlessness, tension or irritability about, e.g., health, work, money or family, for no clear reason. An anxiety disorder may be accompanied by tachycardia or dyspnea. Exemplary anxiety disorders include anxiety, panic disorder with or without agoraphobia, agoraphobia without history of panic disorder, specific phobia (simple phobia), social phobia (social anxiety disorder), obsessive-compulsive disorder (OCD), post-traumatic stress disorder, acute stress disorder, generalized anxiety disorder and anxiety disorder not otherwise specified.

At present, the fourth edition of the Diagnostic and Statistical Manual of Mental Disorders (DSM-IV.TC.) (American Psychiatric Association, Washington, D.C.), provides a diagnostic tool including anxiety and related disorders. These include: panic disorder with or without agoraphobia, agoraphobia without history of panic disorder, specific phobia (simple phobia), social phobia (social anxiety disorder), obsessive-compulsive disorder (OCD), post-traumatic stress disorder, acute stress disorder, generalized anxiety disorder and anxiety disorder not otherwise specified.

The skilled artisan will recognize that there are alternative nomenclatures, nosologies, and classification systems for neurological and psychiatric disorders, and particular anxiety, and that these systems evolve with medical scientific progress. Thus, the term “anxiety disorder” is intended to include like disorders that are described in other diagnostic sources.

In the combination of the present invention, the components 1 and 2 may be administered separately or together in one pharmaceutical composition. In addition, the administration of one element of the combination of the present invention may be prior to, concurrent to, or subsequent to the administration of the other element of the combination.

The elements of the combination of 1 and 2 may be administered by oral, parenteral (e.g., intramuscular, intraperitoneal, intravenous or subcutaneous injection, or implant), buccal, nasal, vaginal, rectal, sublingual, or topical (e.a. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions for the administration of the components 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical compositions containing the active ingredients 1 and 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hardgelatin or HPMC capsules wherein the active ingredient 1 or 2, separately or together, is mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredients 1 and 2, separately or together, in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical compositions of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical compositions containing 1 and 2, separately or together, may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

Preparations according to this invention containing 1 and 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the combinations of this invention containing 1 and 2, separately or together, may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredients 1 and 2 be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

Within the instant invention flibanserin 1 is preferably administered in such an amount that per single dosage between 5 to 200 mg of flibanserin 1 are applied. Preferred are ranges of between 10 to 150 mg, particular preferred 20 to 100 mg of flibanserin 1. Suitable dosage forms may contain for instance 20, 25, 30, 35, 40, 45, 50, 55, 60, 65, 70, 75, 80, 85, 90, 95 or 100 mg of flibanserin 1. The aforementioned values are based on flibanserin 1 in form of the free base. If flibanserin 1 is applied in form of one of its acid addition salts, the corresponding values are readily calculable from the aforementioned values.

Within the instant invention the additional anxiolytic 2 is preferably administered in such an amount that per day between 0.1 to 5000 mg of 2 are applied. Preferred are ranges of between 0.5 to 3500 mg.

In case of the preferred anxiolytic 2 buspirone preferred doses per day are in the range of about 5 to 60 mg, preferably 10 to 50 mg, more preferably 15 to 40 mg. In case of the preferred anxiolytic 2 fluoxetine preferred doses per day are in the range of about 10 to 100 mg, preferably 15 to 80 mg, more preferably 20 to 60 mg. Suitable dosage forms may contain for instance 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445 or 450, 475, 500, 525, 550, 575, 600, 625, 650, 675, 700, 725, 750, 775, 800, 825, 850, 875, 900, 925, 950, 975 or 1000 mg of 2. Advantageously, the compounds 2 of the present invention may be administered in a single daily dose, or the total daily dosage may be administered in divided doses of two, three or four times daily.

In another preferred embodiment the invention relates to a method for the treatment and/or prevention of anxiety disorders, comprising the administration of a therapeutically effective amount of 1 optionally in form of the free base, the pharmacologically acceptable acid addition salts and/or optionally in form of the hydrates and/or solvates thereof, in combination with a therapeutically effective amount of the anxiolytic 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of anxiety disorders selected from the group consisting of anxiety, panic disorder with or without agoraphobia, agoraphobia without history of panic disorder, specific phobia (simple phobia), social phobia (social anxiety disorder), obsessive-compulsive disorder (OCD), post-traumatic stress disorder, acute stress disorder, generalized anxiety disorder and anxiety disorder not otherwise specified comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of anxiety, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of panic disorder with or without agoraphobia, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of agoraphobia without history of panic disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of specific phobia (simple phobia) comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of social phobia (social anxiety disorder) comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of obsessive-compulsive disorder (OCD), comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of post-traumatic stress disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of acute stress disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of generalized anxiety disorder, comprising the administration of a therapeutically effective amount of 1 optionally in form of the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

In another preferred embodiment the invention relates to a method for the treatment and/or prevention of anxiety disorder not otherwise specified, comprising the administration of a therapeutically effective amount of 1 optionally in form the free base, the pharmacologically acceptable acid addition salts and/or optionally in form of the hydrates and/or solvates thereof, in combination with a therapeutically effective amount of 2, optionally in form of the pharmaceutically acceptable acid addition salts, in form of the hydrates and/or solvates and optionally in the form of the individual optical isomers, mixtures of the individual enantiomers or racemates thereof, separately or together within one pharmaceutical composition.

The beneficial effects of the compositions according to the invention can be observed regardless of whether the disturbance existed lifelong or was acquired, and independent of etiologic origin (organic—both, physically and drug induced-, psychogen, a combination of organic—both, physically and drug induced-, and psychogen, or unknown).

Another preferred embodiment of the invention is directed to the aforementioned methods wherein 2 is selected from the group consisting of benzodiazepine agonists, 5-HT_{1A} agonists, 5-HT reuptake inhibitors, chloride channel modulators, MAO-A inhibitors, GABA antagonists, 5-HT2 antagonists, Monoamine reuptake inhibitors, GABA agonists, 5-HT2C antagonists, GABA-A modulators, NK1 antagonists, 5-HT1B antgonists, alpha 2 adrenoreceptor agonists, glutamate agonists, Melatonin agonists, mGluR3 agonists, mGluR2 agonists, CCK2 antagonists, NK 3 antagonists and CGRP antagonists.

Another preferred embodiment of the invention is directed to the aforementioned methods wherein 2 is selected from the group consisting of bupropion, clonazepam, alprazolam, lorazepam, clorazepate, oxazepam, flurazepam, diazepam, halazepam, prazepam, chlordiazepoxide, buspirone, gepirone, tandospirone, ipsapirone, bentazepam, citalopram, clobazam, clotiazepam, etifoxine, etizolam, delorazepam, ethyl loflazepate, flutazolam, fluoxetine, flutoprazepam, ketazolam, metaclazepam, mexazolam, moclobemide, oxazolam, tofisopam, pinazepam, paroxetine, pivagabine, rilmazafone, sertraline, tianeptine, venlafaxine, zotepine, escitalopram, fluvoxamine, pregabalin (PD-144723), agomelatine, duloxetine, LY-544344 (Elly Lilly), ocinaplon, pagoclone, aprepitant, dexmedetomidine, eglumegad, POL-240 (1H-indole-3-pyruvic acid), eplivanserin, vestipitant, levetiracetam,

MKC-242 (Medici Nova), olanzapine, R-673 (Roche), SL-651498 (Sanofi-Aventis), SLV-308 (Solvay), tiagabine, ABT-089 (Abbott), emapunil, dextofisopam, itriglumide, S-desmethylzopiclone, SSR-146977 (Sanofi-Aventis), SSR-149415 (Sanofi-Aventis), gabapentin, opipramol, sumatriptan, TPA-023 (L 838417, Merck & Co.) and nefazodone, optionally in form of the pharmaceutically acceptable acid addition salts, in form of the hydrates and/or solvates and optionally in the form of the individual optical isomers, mixtures of the individual enantiomers or racemates thereof.

Another preferred embodiment of the invention is directed to the aforementioned methods wherein 2 is selected from the group consisting of fluoxetine and buspirone, optionally in form of the pharmaceutically acceptable salts, in form of the hydrates and/or solvates and optionally in the form of the individual optical isomers, mixtures of the individual enantiomers or racemates thereof.

Another embodiment of the invention relates to the use of the combinations of 1, optionally in form the free base, the pharmacologically acceptable acid addition salts and/or optionally in form of the hydrates and/or solvates thereof, and one or more additional anxiolytics 2, optionally in form of the pharmaceutically acceptable salts, in form of the hydrates and/or solvates and optionally in the form of the individual optical isomers, mixtures of the individual enantiomers or racemates thereof, for the preparation of a medicament for the treatment and/or prevention of the aforementioned disorders.

Another embodiment of the invention relates to the use of the combinations of 1, optionally in form the free base, the pharmacologically acceptable acid addition salts and/or optionally in form of the hydrates and/or solvates thereof, and 2, optionally in form of their pharmaceutically acceptable acid addition salts for the preparation of a medicament for the treatment and/or prevention of the aforementioned disorders, wherein 2 is selected from the group consisting of benzodiazepine agonists, 5-HT_{1A} agonists, 5-HT reuptake inhibitors, Chloride channel modulators, MAO-A inhibitors, GABA antagonists, 5-HT2 antagonists, Monoamine reuptake inhibitors, GABA agonists, 5-HT2C antagonists, GABA-A modulators, NK1 antagonists, 5-HT1B antgonists, alpha 2 adrenoreceptor agonists, glutamate agonists, Melatonin agonists, mGluR3 agonists, mGluR2 agonists, CCK2 antagonists, NK 3 antagonists and CGRP antagonists.

Another embodiment of the invention relates to the use of the combinations of 1, optionally in form the free base, the pharmacologically acceptable acid addition salts and/or optionally in form of the hydrates and/or solvates thereof, and 2, optionally in form of their pharmaceutically acceptable acid addition salts for the preparation of a medicament for the treatment and/or prevention of the aforementioned disorders, wherein 2 is selected from the group consisting of bupropion, clonazepam, alprazolam, lorazepam, clorazepate, oxazepam, flurazepam, diazepam, halazepam, prazepam, chlordiazepoxide, buspirone, gepirone, tandospirone, ipsapirone, bentazepam, citalopram, clobazam, clotiazepam, etifoxine, etizolam, delorazepam, ethyl loflazepate, flutazolam, fluoxetine, flutoprazepam, ketazolam, metaclazepam, mexazolam, moclobemide, oxazolam, tofisopam, pinazepam, paroxetine, pivagabine, rilmazafone, sertraline, tianeptine, venlafaxine, zotepine, escitalopram, fluvoxamine, pregabalin (PD-144723), agomelatine, duloxetine, LY-544344 (Elly Lilly), ocinaplon, pagoclone, aprepitant, dexmedetomidine, eglumegad, POL-240 (1H-indole-3-pyruvic acid), eplivanserin, vestipitant, levetiracetam, MKC-242 (Medici Nova), olanzapine, R-673 (Roche), SL-651498 (Sanofi-Aventis), SLV-308 (Solvay), tiagabine, ABT-089 (Abbott), emapunil, dextofisopam, itriglumide, S-desmethylzopiclone, SSR-146977 (Sanofi-Aventis), SSR-149415 (Sanofi-Aventis), gabapentin, opipramol, sumatriptan, TPA-023 (L 838417, Merck & Co.) and nefazodone, optionally in form of the pharmaceutically acceptable acid addition salts, in form of the hydrates and/or solvates and optionally in the form of the individual optical isomers, mixtures of the individual enantiomers or racemates thereof.

Another embodiment of the invention relates to the use of the combinations of 1, optionally in form the free base, the pharmacologically acceptable acid addition salts and/or optionally in form of the hydrates and/or solvates thereof, and 2, optionally in form of the pharmaceutically acceptable salts, in form of the hydrates and/or solvates and optionally in the form of the individual optical isomers, mixtures of the individual enantiomers or racemates thereof, for the preparation of a medicament for the treatment and/or prevention of the aforementioned disorders, wherein 2 is selected from the group consisting of fluoxetine and buspirone.

The following examples demonstrate possible pharmaceutical compositions comprising flibanserin in combination with one of the aforementioned combination partners 2.

### EXAMPLE NO 1

### Combination 1 with Fluoxetine

| Constituents | mg/tablet |
|---|---|
| Core | |
| Flibanserin (free base) | 50.000 |
| Fluoxetine hydrochloride | 22.400 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |
| Coating | |
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 402.175 |

### EXAMPLE NO 2

### Combination 1 with Buspirone

| Constituents | mg/tablet |
|---|---|
| Core | |
| Flibanserin (free base) | 50.000 |
| Buspirone hydrochloride | 5.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |
| Coating | |
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 395.000 |

### EXAMPLE NO 3

### Combination 1 with Alprazolam

| Constituents | mg/tablet |
|---|---|
| Core | |
| Flibanserin (free base) | 50.000 |
| Alprazolam | 1.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |
| Coating | |
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 357.000 |

### EXAMPLE NO 4

### Combination of 1 with Citalopram

| Constituents | mg/tablet |
|---|---|
| Final Mixture | |
| Flibanserin (free base) | 50.000 |
| Citalopram hydrobromide | 49.980 |
| Lactose monohydrate | 200.000 |
| Pregelatinized starch | 108.000 |
| Magnesium stearate | 2.000 |
| Capsule | |
| Final Mixture | 409.980 |
| Capsule (size 1) | 82.000 |
| Total weight of Capsule | 491.980 |

The following examples show preferred pharmaceutical compositions of flibanserin, if the combinations according to the invention are administered in separate dosage units.

### EXAMPLE NO 5

### Composition

| Constituents | mg/tablet |
|---|---|
| Core | |
| Flibanserin (free base) | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### EXAMPLE NO 6

### Composition

| Constituents | mg/tablet |
|---|---|
| Core | |
| Flibanserin (free base) | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |
| Coating | |
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### EXAMPLE NO 7

### Composition

| Constituents | mg/tablet |
|---|---|
| Core | |
| Flibanserin (free base) | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |
| Coating | |
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### EXAMPLE NO 8

### Composition

| Constituents | mg/tablet |
|---|---|
| Core | |
| Flibanserin (free base) | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### EXAMPLE NO 9

### Composition

| Constituents | mg/tablet |
|---|---|
| Core | |
| Flibanserin (free base) | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### EXAMPLE NO 10

### Composition

| Constituents | mg/tablet |
|---|---|
| Core | |
| Flibanserin (free base) | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

What is claimed is:

1) A pharmaceutical composition comprising a therapeutically effective amount of flibanserin, in the form of a free base or a pharmacologically acceptable acid addition salt, in combination with a therapeutically effective amount of an additional anxiolytic.

2) The pharmaceutical composition according to claim 1, wherein the additional anxiolytic is selected from the group consisting of benzodiazepine agonists, 5-HT_{1A} agonists, 5-HT reuptake inhibitors, Chloride channel modulators, MAO-A inhibitors, GABA antagonists, 5-HT2 antagonists, Monoamine reuptake inhibitors, GABA agonists, 5-HT2C antagonists, GABA-A modulators, NK1 antagonists, 5-HT1B antagonists, alpha 2 adrenoreceptor agonists, glutamate agonists, Melatonin agonists, mGluR3 agonists, mGluR2 agonists, CCK2 antagonists, NK 3 antagonists and CGRP antagonists.

3) The pharmaceutical composition according to claim 1, wherein the additional anxiolytic is selected from the group consisting of bupropion clonazepam, alprazolam, lorazepam, clorazepate, oxazepam, flurazepam, diazepam, halazepam, prazepam, chlordiazepoxide, buspirone, gepirone, tandospirone, ipsapirone, bentazepam, citalopram, clobazam, clotiazepam, etifoxine, etizolam, delorazepam, ethyl loflazepate, flutazolam, fluoxetine, flutoprazepam, ketazolam, metaclazepam, mexazolam, moclobemide, oxazolam, tofisopam, pinazepam, paroxetine, pivagabine, rilmazafone, sertraline, tianeptine, venlafaxine, zotepine, escitalopram, fluvoxamine, pregabalin (PD-144723), agomelatine, duloxetine, LY-544344, ocinaplon, pagoclone, aprepitant, dexmedetomidine, eglumegad, POL-240 (1H-indole-3-pyruvic acid), eplivanserin, vestipitant, levetiracetam, MKC-242, olanzapine, R-673, SL-651498, SLV-308, tiagabine, ABT-089, emapunil, dextofisopam, itriglumide, S-desmethylzopiclone, SSR-146977, SSR-149415, gabapentin, opipramol, sumatriptan, TPA-023 (L 838417) and nefazodone.

4) The pharmaceutical composition according to claim 1, wherein the additional anxiolytic is selected from the group consisting of fluoxetine and buspirone.

5) The pharmaceutical composition according to claim 1, wherein flibanserin, in the form of a free base or a pharmacologically acceptable acid addition salt, and the additional anxiolytic are together in one dosage form.

6) The pharmaceutical composition according to claim 1, wherein flibanserin, in the form of a free base or a pharmacologically acceptable acid addition salt, and the additional anxiolytic are separate, each in one dosage form.

7) The pharmaceutical composition of claim 1, wherein flibanserin, in the form of a free base or a pharmacologically acceptable acid addition salt, is a hydrate and/or a solvate.

8) The pharmaceutical composition of claim 1, wherein the additional anxiolytic is in the form of a pharmaceutically acceptable acid addition salt.

9) The pharmaceutical composition of claim 1, wherein the additional anxiolytic is a hydrate and/or a solvate.

10) The pharmaceutical composition of claim 1, wherein the additional anxiolytic is an individual optical isomer, a mixture of individual enantiomers or racemates thereof.

11) A method for the treatment and/or prevention of an anxiety disorder, comprising the administration of a therapeutically effective amount of flibanserin, in the form of a free base or a pharmacologically acceptable acid addition salt, in combination with a therapeutically effective amount of an additional anxiolytic, wherein the flibanserin, in the form of a free base or a pharmacologically acceptable acid addition salt, and the additional anxiolytic are administered separately, each in one dosage form, or together, within one dosage form.

12) The method according to claim 11, wherein the anxiety disorder is selected from anxiety, panic disorder with or without agoraphobia, agoraphobia without history of panic disorder, specific phobia (simple phobia), social phobia (social anxiety disorder), obsessive-compulsive disorder (OCD), post-traumatic stress disorder, acute stress disorder, generalized anxiety disorder and other anxiety disorders.

13) The method according to claim 11, wherein the additional anxiolytic is selected from the group consisting of benzodiazepine agonists, 5-HT_{1A} agonists, 5-HT reuptake inhibitors, Chloride channel modulators, MAO-A inhibitors, GABA antagonists, 5-HT2 antagonists, Monoamine reuptake inhibitors, GABA agonists, 5-HT2C antagonists, GABA-A modulators, NK1 antagonists, 5-HT1B antgonists, alpha 2 adrenoreceptor agonists, glutamate agonists, Melatonin agonists, mGluR3 agonists, mGluR2 agonists, CCK2 antagonists, NK 3 antagonists and CGRP antagonists.

14) The method according to claim 11, wherein the additional anxiolytic is selected from the group consisting of clonazepam, alprazolam, lorazepam, clorazepate, oxazepam, flurazepam, diazepam, halazepam, prazepam, chlordiazepoxide, buspirone, gepirone, tandospirone, ipsapirone, bentazepam, citalopram, clobazam, clotiazepam, etifoxine, etizolam, delorazepam, ethyl loflazepate, flutazolam, fluoxetine, flutoprazepam, ketazolam, metaclazepam, mexazolam, moclobemide, oxazolam, tofisopam, pinazepam, paroxetine, pivagabine, rilmazafone, sertraline, tianeptine, venlafaxine, zotepine, escitalopram, fluvoxamine, pregabalin (PD-144723), agomelatine, duloxetine, LY-544344, ocinaplon, pagoclone, aprepitant, dexmedetomidine, eglumegad, POL-240 (1H-indole-3-pyruvic acid), eplivanserin, vestipitant, levetiracetam, MKC-242, olanzapine, R-673, SL-651498, SLV-308, tiagabine, ABT-089, emapunil, dextofisopam, itriglumide, S-desmethylzopiclone, SSR-146977, SSR-149415, gabapentin, opipramol, sumatriptan, TPA-023 (L 838417) and nefazodone.

15) The method according to claim 11, wherein the additional anxiolytic is selected from the group consisting of fluoxetine and buspirone.
